(19) United States
(12) Patent Application Publication
(10) Pub. No.: US 2023/0404413 A1
(43) Pub. Date: Dec. 21, 2023

(54) METHOD OF ESTIMATION OF THE HEMODYNAMIC STATUS AND CAPACITY OF EFFORT FROM PATIENTS WITH CARDIOVASCULAR AND PULMONAR DISEASES

(71) Applicant: Andres Nicolas Atamañuk, CABA (AR)

(72) Inventor: Andres Nicolas Atamañuk, CABA (AR)

(21) Appl. No.: 18/205,112

(22) Filed: Jun. 2, 2023

Related U.S. Application Data
(63) Continuation of application No. 17/034,000, filed on Sep. 28, 2020.
(60) Provisional application No. 63/082,033, filed on Sep. 23, 2020.
(60) Provisional application No. 62/906,872, filed on Sep. 27, 2019.

(51) Int. Cl. A61B 5/0205; A61B 5/029; A61B 5/00; G16H 10/20; G16H 30/40; G16H 50/30; G16H 50/20; G16H 50/70; A61B 5/01

(52) U.S. Cl. CPC A61B 5/02055; A61B 5/029; A61B 5/6852; A61B 5/7267; G16H 10/20; G16H 30/40; G16H 50/30; G16H 50/20; G16H 50/70; A61B 5/015; A61B 2576/02; A61B 5/0075; A61B 5/14542

(57) ABSTRACT

The invention described herein is a method that can predict: according to a Modality A, the effort capacity of a human individual as an expression of distance walked by the individual if subjected to a 6MWT; and according to Modality B, the hemodynamic state of the patient as an expression of CI and SVR. The method is comprised of four steps: (1) capturing a series of thermal images of the face and hands of a human individual, according to the modality; (2) applying established temperature values for a series of specific spots from the face and the hands, according to the modality; (3) selecting general additional parameters, from the patient and the environment; and (4) implementing algorithms discovered through the ML technique, through which the previously mentioned parameters are analyzed

### CROSS REFERENCE TO RELATED APPLICATIONS

This application claims the benefit of U.S. Provisional Application No. 62/906,872 filed on Sep. 27, 2019, and U.S. Provisional Application No. 63/082,033 filed on Sep. 23, 2020. The entire contents of this application is incorporated herein by reference in its entirety.

### INVENTION FIELD

The invention described herein belongs to the field of clinical methodologies and analysis to test the state of patients who might require it. In particular, it is related to the methodologies of clinical application which allow diagnosis of the patient's condition; more specifically, it is related to non-invasive methods with the purpose of diagnosing the hemodynamic state or the effort capacity in cardiac and non-cardiac patients.

### STATE-OF-THE-ART DESCRIPTION

The estimation of a patient's hemodynamic state or effort capacity is of the utmost importance since it allows for diagnosis, establishment of prognosis and/or adjustment and evaluation of the effects of a treatment.

The pulmonary artery catheter (PAC), otherwise known as Swan-Ganz catheter, was employed for the first time in 1944. This technique became the main method for measuring cardiac output, reaching the peak of its utilization in the 1980's. Afterwards, the utilization of the PAC has been the subject of intense debate, mainly due to the publication of scientific research in which its utilization was not linked to any benefit in survival and, furthermore, in some of those documents it was associated with a higher mortality rate on account of the risk of iatrogenesis. This process has led to its use diminishing since the 1990's, although at present it is still considered a referential standard technique (gold standard) for hemodynamic testing and it is still employed currently.

Throughout history there has been development of less invasive methods, which are easier to employ and act as surrogates to the gold standard method or provide indirect information such as effort capacity; examples of these methods are given as follows.

Techniques for analysis of arterial pressure contour, whose function requires insertion into an arterial catheter, generally into a peripheric artery such as the radial or the femoral arteries, after which the catheter is connected to a pressure transducer. This group of techniques is based on the principle that the pulse pressure, i.e. difference between the systolic and diastolic blood pressure, is proportional to the systolic volume and inversely proportional to the aortic distensibility. In the transformation into cardiac output there are more than ten different algorithms, all of them complex, based on several mathematic models.

Furthermore, the techniques that utilize arterial pressure contour are differentiated according to their periodicity and the type of calibration required, since some of them require manual calibration whilst others don't require external calibration, the location of arterial cannulation, the parameters analyzed nor the accuracy with which they determine the cardiac output.

The first commercialized technique was the PiCCO® in 1997 (Pulsion, Munich, Germany), which employed the Swan-Ganz catheter and transpulmonary thermodilution for its calibration, utilizing the catheter in the femoral artery. Afterwards, the commercialization of other techniques was authorized, such as the LiDCO® (LiDCO Ltd., Cambridge, England) in the year 2002, and the FloTrac®-Vigileo° (Edwards Lifesciences, Irvine, CA, USA) in 2004. The calibration of the PiCCO and LiDCO techniques is performed through thermodilution and lithium, respectively.

Regarding the mathematical models of the blood pressure contour systems, the FloTrac-Vigileo (Edwards Lifesciences) applies the empirical model based on the pressure of the pulse and the vascular tone; it also employs other data such as arterial pressure, age, sex and body surface area. The PiCCO utilizes the Windkessel model which measures the area below the pressure curve. The LiDCO has an approach which is similar to PiCCO but employs the root mean square, proceeding from the pressure curve. The MostCare® system from Vygon (Vytech, Padova, Italy) employs the Pressure Recording Analytical Method (PRAM), which is based on a modified version of the Wesselings algorithm for arterial pulse wave analysis. Before every cardiac output measurement, the arterial pulse signal is analyzed. This technique differentiates itself from other algorithms of pulse contour analysis that calculate the area under the curve of the pulse wave, both in the systole and the diastole and these parameters are employed for obtaining systemic arterial impedance measurements, between other hemodynamic parameters. The cardiac output is presented as the average value of 12 beats.

There are several non-invasive methods available, for example, impedance cardiography (Thoracic Electrical Bioimpedance) or bio-impedance, which are based on the Ohm law, which determines changes in the thoracic liquid content supported by the changes in the conductivity/resistance to the propagation of an electric impulse through the thorax. A patient is required to have a normal thorax and neck anatomy for electrode placement. The calculation is made from the conduction speed of an electric stimulus and the resistance to the application in the patient. Among its limitations is the fact that, for cardiac output calculation, it takes into account hemodynamic stability values and different artificial sources exist. With the employment of impedance cardiography we can obtain the following parameters: cardiac output (CO), systolic volume (SV), systemic vascular resistance (SVR), ventricular contractibility parameters and blood volume standards, i.e. thoracic fluid content. The fluctuations in voltage and in thoracic electric impedance are fundamentally thanks to the blood flow variations in the large vessels and they're translated into the hemodynamic parameters described above.

The cardiovascular performance or effort capacity can be measured through Cardiopulmonary Exercise Testing (CPET). Though it could be considered the gold standard to evaluate the physiological integration to the exercise of the cardiovascular, respiratory, metabolic, musculoskeletal and neurosensory systems, it has a limitation: it is a technically difficult test, it requires qualified personnel, it is expensive and the duration of the test is rather. In daily clinical practice it is frequently replaced by another test which is easier to conduct, such as the Six-Minute Walk Test (6MWT) or simply through the physical testing of the patient.

Every one of the aforementioned methods is less invasive, but they're still far from being considered practical in their implementation, since they require qualified personnel and even some of them present low levels of correlation against the reference method (Swan-Ganz catheter).

With the current technological advances, the search for methods which are easier in terms of implementation, with instant measurements and a proper correlation with the hemodynamic state or effort capacity of the patients has grown.

### SUMMARY OF THE INVENTION

Therefore, the object of the invention described herein is a method of clinical testing that is non-invasive and provides immediate results to determine the hemodynamic state and/or the effort capacity of a patient under clinical trial.

The method is based on the physiological fact that the temperature present in any region or point on the skin of a person is given by the amount of blood that gets to that region, called perfusion, which depends on the blood flow and the level of vasoconstriction or vasodilatation present in the organs or tissues from that body region, and the metabolic level of the organs or tissues covered by the skin in that region. Perfusion depends largely on cardiac activity, i.e. cardiac output.

Under this precept, in daily practice and through medical examination, the doctor uses their hands to compare the temperature of the patients' skin in different regions, and thus determines the cardiovascular performance. It is understood that if during the physical examination the patient experiences a drop in the temperature in their extremities or distal regions, namely hands, feet and knees, this should be considered as a possible hemodynamic deficit.

The present method consists of capturing thermal images of the patient's face and extremities, thus obtaining the temperature values for a series of specific points in established areas that have already been determined by research and testing, that is thermal averages and thermal gradients between different spots, and also incorporating into the examination other additional general parameters from the patient and the environment, and processing said data through a computational algorithm that utilizes a predictive model employing Machine Learning (ML) tools.

The data acquired as a result of this process allows for a series of predictions to be made: a) the distance that a patient could complete if subjected to a 6MWT, called ‘Modality A’, the estimation of a patient's effort capacity, and b) Cardiac Index (CI) and Systemic Vascular Resistance (SVR), called ‘Modality B’, the patient's hemodynamic state.

Preferably, the temperature values are determined through thermal images of the front and profile of the individual's face and palms. These images can be taken by any infrared thermographic camera. The invention employs a camera which captures infrared images and allows for the data of interest to be obtained immediately: according to Modality A ; the distance walked in meters if subjected to a 6MWT; and according to Modality B, the Cardiac Index informed as patient WITHOUT heart failure or patient WITH heart failure (cutting point 2.5 liters/min/m²) and/or Systemic Vascular Resistance, informed as high or low SVR (reference value of 1000 dines/sec.cm⁻⁵).

For ‘Modality B’, it is preferable that the temperature values are determined in both eyes (OP), interciliar region (ICF), the nose root (NRF) and the inferior part of the dihedral angle formed between the nose and the infraorbital region (ADEC). For ‘Modality A’, in the eyes (right, Od; left, Oi), the nose (Na), the auditor conduct (Ca) and the pinna (Pa) in the thermal images of the front and profile of the individual's face and their palm (palmar face) as well as the wrist, central region of the palm and extremes of the fingers.

It is preferable to establish averages and thermal gradients between the temperature values.

It is even more preferable that, for ‘Modality A’ the thermal gradients are established between the Od value and the Na (NO Gradient) and between the Ca and the Pa (CP Gradient), and between different points of the hand.

Regarding ‘Modality B’, the thermal average employed is calculated between ADEC-ICF-NRF; and the gradient between ADEC-OP and OP thermal gradient between ADEC-ICF.

Additionally, the general parameters of the patient for ‘Modality A’ are the following: age, gender, size, weight, cardiac frequency, arterial pressure, functional class, oxygen saturation and any vasodilation drugs that the patient might be taking. For the ‘Modality B’ the parameters are: gender, central venous pressure values, administration, or not, of vasoactive drugs, namely vasoconstrictor or vasodilator drugs or both, waking state or pharmacological sedation.

Moreover, the general additional parameters related to the environment are humidity and room temperature.

### BRIEF DESCRIPTION OF THE FIGURES

The features and advantages of the present invention will become more readily apparent from the following detailed description of the invention in which like elements are labeled similarly and in which:

FIGS. 1A, 1B, and 1C are a series of thermal images according to Modality A of the invention, in which the circles encompass the areas of interest to be measured, the arrows that indicate white or brighter areas signify the hottest spots and the arrows that indicate the darker areas signify the coldest ones. FIG. 1A shows an infrared thermal image of the front of an individual's face. FIG. 1B shows an infrared thermal image of the profile of an individual's face. FIG. 1C shows an infrared thermal image of an individual's palm.

FIGS. 2A and 2B are a series of thermal images according to Modality B of the invention. They demonstrate the face of a patient, from a frontal view. In FIG. 2A, the picture on the left, the different areas of interest are shown, whereby it is possible to identify the thermal points to be measured: the arrows pointing upwards belong to the hottest spots; the arrows pointing downwards belong to the coldest spots. In FIG. 2B, the photo on the right, arrow number 2 points out the highest thermal areas and temperatures of the eyes (OP); arrow number 1 points out the area with the lowest temperature of the ICF. Arrow number 3 marks the lowest NRF temperature and arrow number 4 indicates the hottest ADEC spot.

FIG. 3 shows the way in which thermal images of the face of a patient are obtained, following the method of the invention in Modality A.

FIG. 4 shows the way in which thermal images from the faces of two different patients in a Critical Care Unit were obtained. These patients required invasive monitoring with the Swan-Ganz catheter, according to the method of ‘Modality B’ of the invention. The thermal image obtained is shown below the picture on the right.

FIG. 5 is a schematic representation of the steps of the method of the invention is put into practice in three steps. In the first step the general data such as weight, size, age, environment parameters, etc. is uploaded. In the second step, thermal images from the patient's face are obtained - there must be three pictures per analysis area. In the third step, the desired result is obtained. For this purpose, a smartphone with a thermal camera attached to it could be employed. Note: the pictures show a Samsung A5 cellphone, with a FLIR ONE thermal camera attached.

FIGS. 6A, 6B, 6C, 6D are a series of graphs showing the correlativity between the thermal gradients between the central areas of the eyes and the auditive conduct, and distal regions from nose and auricular pavilion. The graphs, FIGS. 6C, 6D, in the upper part of the figure, present patients with Pulmonary Hypertension (PH), FIG. 6C, the one on the left, takes into account the net distance completed in the 6MWT: r2−0.44, (IC 95% −0.67−0.13) p 0.006, and FIG. 6D, the one on the right takes into account the predicted value in meters walked, according to age, gender, size and weight: r2−0.43, (IC 95% 0.65−0.13) p 0.007. FIGS. 6A, 6B, the graphs underneath, represent the results obtained in healthy subjects.

FIG. 7 is a graph showing the correlation between the results obtained by the method of the invention, according to Modality A, and the 6MWT for the distance in meters walked in 121 cases prospectively analyzed.

FIG. 8 is a graph showing the ROC curve, analyzed for a cutoff point of 440 m in the 6MWT, employing the prediction according to the method of the invention utilizing its Modality A.

FIGS. 9A, 9B are graphs showing a comparison of the correlations between the prediction of distance in meters walked in the 6MWT in healthy subjects, employing the Gibbons equation FIG. 9A, (left), and the method of the invention according to Modality A FIG. 9B, (right).

FIGS. 10A, 10B are graphs showing a comparison of the correlations between the prediction of distance in meters walked in the 6MWT in patients with PH, employing the Gibbons equation (FIG. 10A (left)) and employing the method of the invention according to Modality A (FIG. 10B (right)).

FIG. 11 is a graph showing the ROC curve for the IC prediction, employing the method of the invention according to Modality B.

FIG. 12 is a graph showing the ROC curve for the SVR prediction, employing the method of the invention according to Modality B.

FIG. 13A is a series of thermal images of the three hemodynamic moments from Clinical Case 1. In the scale of grays, white is the highest thermal spectrum (hot) and dark gray or black are the lowest thermal spectrum (cold). The real hemodynamic values, obtained through the Swan-Ganz catheter, are provided under each picture.

FIG. 13B is the same series of thermal images from FIG. 13A, in which, encompassed within the circled areas, the temperatures registered by thermography are provided in degrees centigrade.

FIG. 14 is a series of thermal images of a patient, employing the method of the invention according to Modality A, before completion of the 6MWT. The circles with dotted lines enclose the areas of interest to analyze the temperatures. The picture on the left shows the face from the front; the picture in the center shows the face from its profile and the picture on the right shows the palm of the right hand of the patient.

FIG. 15A is two thermal images of two hemodynamic moments of Clinical Case 3. In the gray scale, white is the highest thermal spectrum (hot) and dark gray or black is the lowest thermal spectrum (cold). The actual hemodynamic values obtained by the Swan-Ganz catheter are provided below each image.

FIG. 15B is the same series of thermal images from FIG. 15A, in which the temperatures registered in degrees centigrade by thermography are provided within the circled areas with dotted lines.

FIG. 16 is a screen shot of the main piece of code to resolve the module in the modality A.

FIG. 17 is a screen shot of the main piece of code to resolve the module in the modality B: Cardiac Index.

FIG. 18 is a screen shot of the main piece of code to resolve the module in the modality B: systemic vascular resistance.

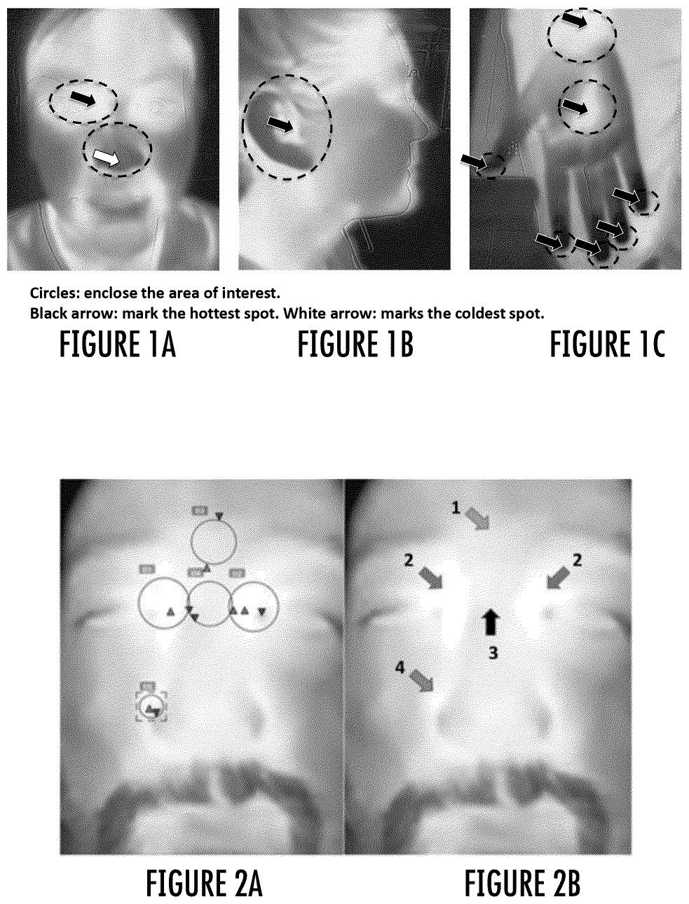

### DETAILED DESCRIPTION OF THE INVENTION

With the purpose of avoiding the inconvenience of previous methods, a methodology consisting of two modalities is proposed:
Modality A: estimation of the patient's effort capacity
Modality B: estimation of the patient's hemodynamic state

Based on the previously stated physiological concept, we understand that the higher the cardiovascular performance, the higher temperature of specific spots on the skin of the face or the extremities. These spots can be found further away from the heart. Since these spots have terminal blood circulation, they are called distal or acral regions. Examples of distal regions are: extremities of the fingers, ears or nose.

Consequently, we can infer the following:
1. The temperature of these ‘distal’ regions can be directly related to the CO and the effort capacity, and inversely related to the SVR of the patient.
2. When comparing the temperature of the distal regions with respect to the proximal regions, a thermal gradient can be established. This thermal gradient between such areas could be inversely proportional to the CO and the effort capacity, and directly proportional to the SVR, since the higher the SVR, the worse the blood circulation.

The register of the temperature in the different spots of interest on the skin from the patients can be obtained through the capture of thermal photos, taken by any thermographic of infrared camera. This camera is a device that, based on the emission of infrared media from the electromagnetic spectrum from the detected bodies, creates luminous images visible to the human eye, i.e. “thermal photographs”.

To put the invention into practice, two types of study were needed. The first one served the purpose of predicting the effort capacity, and the second one that of predicting hemodynamic behavior. Both studies had the purpose of finding which thermal spots and the thermal gradients were related to the aforementioned cardiovascular behaviors.

### First Research

Patients with Pulmonary Hypertension (PH) were analyzed via the use of thermal pictures. PH is a disease that produces heart failure and healthy subjects served as a comparative. After the capturing of the thermal photos, the subjects underwent a 6MWT and the distance completed was measured. The professional in charge of the analysis of the thermal images was unaware (blind′) with regards to the results of the 6MWT.

All of the testing was completed under controlled conditions with regards to temperature, humidity and luminosity, as well as being performed in the same physical place, which served the purpose of ensuring no significant variation in the aforementioned environmental parameters. The devices employed in the testing were a FLIR ONE thermal camera, attached to a SAMSUNG A5 cellphone. Employing the use of the thermal camera, three captures of thermal images were taken of the fact from the frontal view, along with three captures from the profile and three more from the palm of the right hand. An average of the thermal values of each of the three images obtained per region was calculated with the purpose of reducing the margin for error with regards to measurement. Every image was taken keeping the distance between the camera and the region of interest to lesser than 30 cm.

The subjects undergoing the testing had to remain resting in a sitting position in a chair for at least 15 minutes, away from any source of extreme temperatures such as heaters, fridges, fans, air conditioning and the like. The subjects were not able to wear contact lenses, glasses, facial or ear piercings, rings or bracelets, nor any type of adornment that could interfere with the measurement of the skin temperature.

Subjects with skin damage, infections in the breathing area or in their ears, untreated thyroid gland disease or any kind of acute infection were excluded, since these conditions could modify the temperature.

After the capture of the images, the subjects were put under testing and the 6MWT following the recommendations present in any international guidelines that describe and validate such testing, and the distance completed in this test was measured.

The test was experimental and transversal, recruiting patients in a prospective way. 55 cases were analyzed, 39 patients with PH and 16 healthy subjects. The average age of said subjects/patients was 45.74±15.5 years and 84% of the patients were women. Consecutive patients were included, i.e. without going through selection, and the PH diagnosis was defined by international criteria for precapillary PH by right cardiac catheterization. The images obtained were analyzed through a library of code and software provided by the supplier of the thermal camera, which allows for the interpretation of the infrared color spectrum and translation into temperature in degrees centigrade.

Higher temperatures were registered in the following regions: eyes, right auditive conduct and the Pterion region, whereas the lowest temperatures were obtained in the nasal region and the right auditive pavilion.

Regarding the palm of the hand, the registered temperatures were higher in the following regions: wrist, center of the palm and fingertips.

The thermal gradients (TG) were established between NO and CP. The comparative was performed between the TG from PH patients and the TG from the control group.

Moreover, anthropometric data was registered, such as age, type of disease for the group of PH patients, arterial pressure and oxygen saturation. Whether the patients were taking any medication that could generate cutaneous vasodilation was also taken into account.

The numeric variables were expressed as mean and DS. The TG's were compared with the Student Test. Then the Pearson correlation was applied between TG and the distance walked during the 6MWT. A p<0.05 was considered significant.

The following results were obtained in relation to the distance walked in the 6MWT: 386±151 versus 533±88 mts (<0.001) for PH and controls, respectively. See Chart 1 below:

| | | Pre 6MWT | | | Post 6MWT | | |
|---|---|---|---|---|---|---|---|
| | | PH | | | PH | | |
| Areas or regions | | patients | CONTROLS | p | patients | CONTROLS | p |
| FACE, | Right eye | 37.3 ± 2.5 | 37 ± 3.2 | 0.65 | 35.1 ± 2.3 | 35.6 ± 2.9 | 0.41 |
| FRONT | Nose | 30.6 ± 3.4 | 31.6 ± 3.6 | 0.21 | 27 ± 3.5 | 28.2 ± 3.5 | 0.20 |
| (° C.) | Thermal | 6.7 ± 2 | 5.6 ± 2.6 | 0.05 | 8 ± 2.3 | 7.6 ± 2 | 0.42 |
| | Gradient RE-N | | | | | | |
| FACE, | Ear Canal | 38.1 ± 1.6 | 38.1 ± 2.2 | 0.99 | 36.4 ± 2.3 | 36.9 ± 2 | 0.35 |
| SIDE | External Ear | 31 ± 2.4 | 31.3 ± 2.8 | 0.62 | 27.8 ± 3 | 29.5 ± 2.7 | 0.01 |
| (° C.) | Thermal | 7.2 ± 1.5 | 6.9 ± 1.8 | 0.47 | 8.6 ± 1.5 | 7.5 ± 2.7 | 0.02 |
| | Gradient EC- | | | | | | |
| | EE | | | | | | |
| PALM OF | Central Area | 34.4 ± 2.9 | 35.2 ± 2.5 | 0.33 | 31.5 ± 3.2 | 33.1 ± 3.2 | 0.09 |
| THE | 1° finger | 29.5 ± 4.6 | 32 ± 4 | 0.06 | 25.3 ± 4.6 | 29.3 ± 5.3 | 0.008 |
| RIGHT | 2° finger | 28.9 ± 4.4 | 31.6 ± 4.2 | 0.04 | 24.5 ± 4.6 | 28.9 ± 5.3 | 0.003 |
| HAND | 3° finger | 29 ± 4.5 | 31.1 ± 3.9 | 0.12 | 24.4 ± 4.9 | 28 ± 5.4 | 0.02 |
| (° C.) | 4° finger | 28.8 ± 4.4 | 31 ± 4.4 | 0.10 | 24 ± 4.6 | 28.1 ± 5.8 | 0.009 |
| | 5° finger | 28.8 ± 4.5 | 31 ± 4.5 | 0.12 | 23.9 ± 4.6 | 28.1 ± 6.1 | 0.009 |

Chart 1 shows the thermal differences between PH and controls, registered before performing the 6MWT. For a better understanding and evaluation of the method, several thermal images were taken after the 6MWT.

The subjects with PH present a lower temperature in the distal regions in comparison with the healthy controls. Although some thermal differences between both groups can be quantified, some differences were meaningful while others were not. At first approach, this could be explained by the limited number of the sample.

When taking into account the total number of individuals in the test, i.e. those with PH and healthy people, a clearly established and meaningful thermal gradient was found, which proves that distal regions show a lower temperature than the central regions. See Chart 2 below:

| Regions | Basal (pre 6MWT) | | | Final (Pos 6MWT) | | |
|---|---|---|---|---|---|---|
| Com- | Coefficient | | | Coefficient | | |
| pared | β | IC 95 | p | β | IC 95 | p |
| Wrist vs | 0.99 | 0.88-1.1 | <0.001 | 1.02 | 0.9-1.15 | <0.001 |
| RCP | | | | | | |
| RCP vs | 1.39 | 1.2-1.6 | <0.001 | 1.42 | 1.22-1.62 | <0.001 |
| PTD | | | | | | |

Chart 2 shows the figures for the beta coefficients when comparing the three regions before the 6MWT and when completed; wrist vs. central region of the front of the hand (RCP), and RCP vs. Five Fingers Temperature Average (PTD).

The temperature registered in the palm of the hand before the 6MWT showed a thermal gradient descending through the distal regions, with the temperature of the wrist at 35.2±2.7° C., superior to the temperature of the central region from the palm of the hand, 34.8±2.9° C., and the registered average temperature of the five fingers, 29.8±4.6° C. This gradient of temperature, from proximal to distal, was in turn evident when finishing the exercise: wrist 32.4±2.8° C.; central region 32.1±3.2° C.; average of the five fingers 25.7±5° C. All the differences were statistically significant (p<0.001).

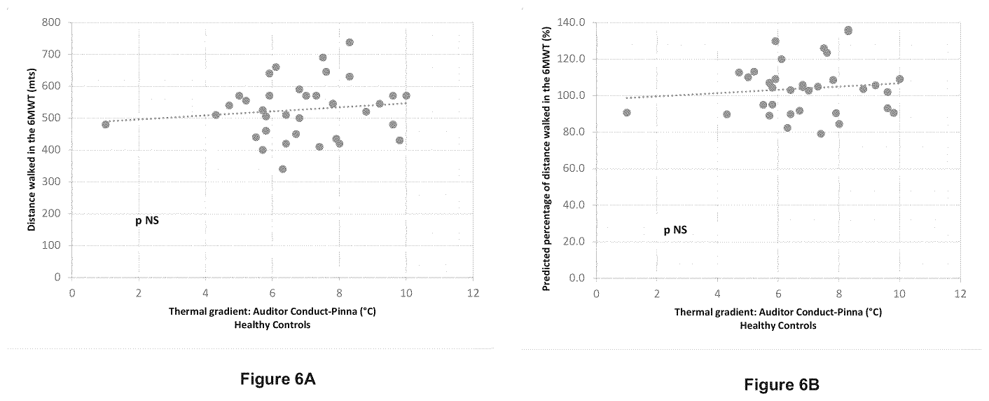

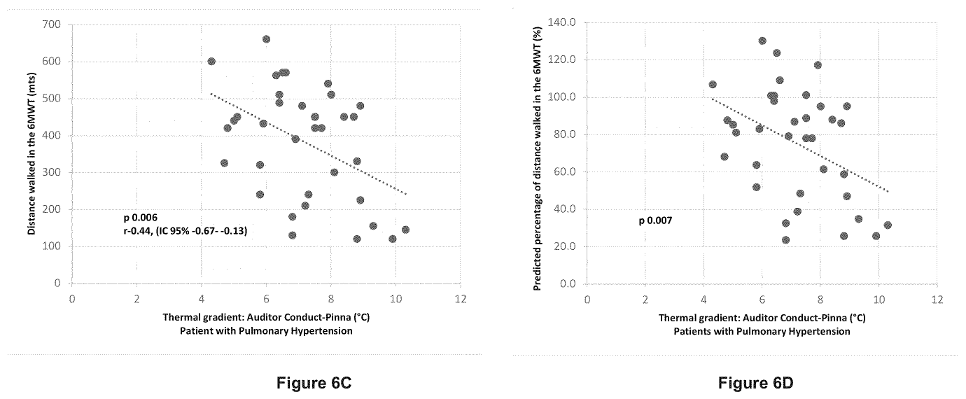

A correlation was determined between the TG and the 6MWT (see FIG. 6).

Consequently, the following was established: (1) the existence of thermal gradients with descending temperature through the distal regions; (2) the existence of a link between thermal gradients and meters walked by the patients during the 6MWT, which explains that, the higher the thermal gradient, the lower the distance completed during the 6MWT; and (3) it was observed that the value of these thermal spots and their behavior during physical exertion is different between ill subjects and healthy ones.

### Second Research

A prospective test was performed on patients hospitalized in Critical Care Units, who had to be monitored in an invasive way by the use of Swan-Ganz catheter.

The age average was 58 years, 70% were men, 28% were sedated and on mechanical respiratory assistance (MRA). 41.6% were taking endovenous vasoconstricting drugs, such as norepinephrine, epinephrine and/or vasopressin; 25% were taking vasodilating drugs, such as milrinone, dobutamine and/or sodium nitroprusside; 25% were taking both types of drugs, and 8.3% none of the aforementioned.

Hemodynamic measurements were performed through Swan-Ganz and the thermodilution method; at the same time, facial thermal images were taken. In every thermal test of the face, i.e. the capture of three images at the same time, the central venous pressure in the right auricle (PVC), the pulmonary interlocking pressure (Pw), systemic average arterial pressure (TAM), cardiac frequency (CF), cardiac minute volume (CMV), cardiac index (CI) and systolic work index of the right/left ventricle (SWIRV, SWILV) were registered.

Thermal images from the front of the patients' face were obtained at a distance of 10 cm and the best links between thermal spots and gradients were researched, along with the majority of the hemodynamic variables already mentioned. The tested thermographic variables were correlated with the Pearson and the Spearman Tests. p<0.05 was considered to be a statistically significant value.

Correlations with significant statistical value were found for both Spearman and Pearson tests between CI and SVR with different thermal spots, but not for other hemodynamic variables, except for that of the PVC.

It was found that these correlations between spots and gradients with CI and SVR had different results and, unexpectedly, we found that they depended mainly on the state of the patient being tested: awake versus sedated, receiving vasodilating drugs vs. vasoconstrictors; with a high vs. low PVC and also according to the gender. See Charts 3, 4 and 5 below:

| Correlation by means of the Pearson Test of the Cardiac Index with different points and thermal gradients | | | | | | | | | | | CVP | |
|---|---|---|---|---|---|---|---|---|---|---|---|---|
| | r2- | | | | | | | | | | r2- | |
| | Total | | | | r2- | | r2- | | r2- | | Total | |
| Temperature | popu- | | r2- | | Sleep- | | Vaso- | | Vaso | | popu- | |
| points | lation | p | Awake | p | ing | p | dilators | p | constrictors | p | lation | p |
| OP | 0.26 | 0.13 | 0.13 | 0.5 | 0.74 | 0.02 | 0.06 | 0.8 | 0.36 | 0.15 | 0.33 | 0.054 |
| OP4 | 0.29 | 0.08 | 0.18 | 0.37 | 0.74 | 0.02 | 0.09 | 0.7 | 0.4 | 0.11 | 0.33 | 0.047 |
| ICF | 0.42 | 0.012 | 0.28 | 0.16 | 0.8 | 0.008 | 0.13 | 0.5 | 0.63 | 0.006 | 0.42 | 0.012 |
| ADEC | 0.41 | 0.014 | 0.4 | 0.04 | 0.68 | 0.04 | 0.36 | 0.14 | 0.48 | 0.05 | 0.35 | 0.035 |
| NRF | 0.37 | 0.027 | 0.43 | 0.028 | 0.75 | 0.018 | 0.29 | 0.23 | 0.51 | 0.037 | 0.37 | 0.029 |
| NPC | 0.33 | 0.05 | 0.38 | 0.05 | 0.44 | 0.23 | 0.36 | 0.14 | 0.31 | 0.22 | 0.2 | 0.24 |
| NPF | 0.31 | 0.06 | 0.36 | 0.06 | 0.45 | 0.22 | 0.35 | 0.15 | 0.29 | 0.25 | 0.22 | 0.19 |
| Pr ADEC-ICF | 0.42 | 0.01 | 0.35 | 0.07 | 0.76 | 0.017 | 0.25 | 0.3 | 0.57 | 0.016 | 0.4 | 0.017 |
| Pr ICF-NRF | 0.42 | 0.013 | 0.4 | 0.04 | 0.79 | 0.011 | 0.25 | 0.31 | 0.58 | 0.014 | 0.41 | 0.013 |
| Pr ADEC-NRF | 0.4 | 0.016 | 0.44 | 0.023 | 0.72 | 0.027 | 0.33 | 0.17 | 0.52 | 0.032 | 0.38 | 0.024 |
| Pr3 ADEC- | 0.42 | 0.011 | 0.41 | 0.034 | 0.76 | 0.017 | 0.28 | 0.24 | 0.56 | 0.018 | 0.4 | 0.015 |
| ICF-NRF | | | | | | | | | | | | |
| Sumatoria | 0.39 | 0.017 | 0.33 | 0.09 | 0.76 | 0.016 | 0.18 | 0.46 | 0.57 | 0.01 | 0.4 | 0.011 |
| X1 | | | | | | | | | | | | |
| Gr OP-ADEC | −0.35 | 0.039 | −0.5 | 0.009 | 0.35 | 0.35 | −0.59 | 0.009 | −0.22 | 0.38 | −0.09 | 0.58 |
| Gr OP-ICF | −0.41 | 0.014 | −0.41 | 0.037 | −0.22 | 0.57 | −0.25 | 0.3 | −0.48 | 0.05 | −0.25 | 0.13 |
| Gr OP-NRF | −0.29 | 0.08 | −0.42 | 0.03 | −0.59 | 0.09 | −0.32 | 0.19 | −0.4 | 0.1 | −0.24 | 0.16 |
| Gr OP-Pr | −0.45 | 0.006 | −0.53 | 0.005 | −0.03 | 0.9 | −0.56 | 0.01 | −0.42 | 0.09 | −0.21 | 0.23 |
| ADEC-ICF | | | | | | | | | | | | |
| Gr OP-Pr ICF- | −0.36 | 0.03 | −0.44 | 0.023 | −0.42 | 0.25 | −0.33 | 0.17 | −0.49 | 0.046 | −0.27 | 0.11 |
| NRF | | | | | | | | | | | | |
| Gr OP-Pr | −0.33 | 0.049 | −0.47 | 0.015 | −0.21 | 0.57 | −0.42 | 0.08 | −0.4 | 0.1 | −0.21 | 0.21 |
| ADEC-NRF | | | | | | | | | | | | |
| Gr3 OP-Pr | −0.38 | 0.022 | −0.48 | 0.013 | −0.25 | 0.5 | −0.42 | 0.08 | −0.47 | 0.056 | −0.25 | 0.15 |
| ADEC-ICF- | | | | | | | | | | | | |
| NRF | | | | | | | | | | | | |
| Correlation by means of the Spearman Test of the Cardiac Index with different points and thermal gradients | | | | | | | | | | | CVP | |
| | r2- | | | | | | | | | | r2- | |
| | Total | | | | r2- | | r2- | | r2- | | Total | |
| Temperature | popu- | | r2- | | Sleep- | | Vaso- | | Vaso | | popu- | |
| points | lation | p | Awake | p | ing | p | dilators | p | constrictors | p | lation | p |
| OP | 0.16 | 0.34 | 0.13 | 0.52 | 0.83 | 0.005 | 0.04 | 0.88 | 0.33 | 0.19 | 0.37 | 0.027 |
| OP4 | 0.22 | 0.2 | 0.2 | 0.31 | 0.81 | 0.007 | 0.08 | 0.74 | 0.35 | 0.16 | 0.38 | 0.021 |
| ICF | 0.3 | 0.07 | 0.17 | 0.38 | 0.79 | 0.01 | 0.006 | 0.97 | 0.64 | 0.005 | 0.42 | 0.011 |
| ADEC | 0.3 | 0.07 | 0.33 | 0.09 | 0.81 | 0.007 | 0.28 | 0.25 | 0.45 | 0.06 | 0.34 | 0.044 |
| NRF | 0.32 | 0.058 | 0.38 | 0.054 | 0.81 | 0.007 | 0.2 | 0.41 | 0.49 | 0.04 | 0.4 | 0.016 |
| NPC | 0.28 | 0.09 | 0.35 | 0.07 | 0.55 | 0.11 | 0.38 | 0.11 | 0.26 | 0.3 | 0.24 | 0.15 |
| NPF | 0.2 | 0.11 | 0.34 | 0.08 | 0.59 | 0.09 | 0.35 | 0.15 | 0.24 | 0.34 | 0.26 | 0.12 |
| Pr ADEC-ICF | 0.29 | 0.08 | 0.24 | 0.23 | 0.85 | 0.0039 | 0.12 | 0.6 | 0.58 | 0.013 | 0.4 | 0.016 |
| Pr ICF-NRF | 0.28 | 0.09 | 0.31 | 0.12 | 0.85 | 0.0039 | 0.13 | 0.58 | 0.53 | 0.027 | 0.43 | 0.01 |
| Pr ADEC-NRF | 0.31 | 0.06 | 0.35 | 0.07 | 0.81 | 0.007 | 0.18 | 0.46 | 0.48 | 0.051 | 0.37 | 0.029 |
| Pr3 ADEC- | 0.31 | 0.06 | 0.33 | 0.09 | 0.85 | 0.0039 | 0.15 | 0.53 | 0.55 | 0.021 | 0.41 | 0.014 |
| ICF-NRF | | | | | | | | | | | | |
| Sumatoria | 0.29 | 0.08 | 0.22 | 0.27 | 0.85 | 0.0039 | 0.04 | 0.86 | 0.61 | 0.009 | 0.45 | 0.0068 |
| X1 | | | | | | | | | | | | |
| Gr OP-ADEC | −0.35 | 0.039 | −0.47 | 0.014 | 0.3 | 0.42 | −0.65 | 0.0037 | 0.26 | 0.3 | −0.11 | 0.5 |
| Gr OP-ICF | −0.27 | 0.11 | 0.26 | 0.18 | −0.01 | 0.96 | −0.01 | 0.93 | −0.35 | 0.16 | −0.2 | 0.27 |
| Gr OP-NRF | −0.33 | 0.053 | −0.41 | 0.034 | −0.37 | 0.32 | −0.29 | 0.23 | −0.44 | 0.07 | −0.31 | 0.06 |
| Gr OP-Pr | −0.39 | 0.019 | −0.42 | 0.03 | 0.1 | 0.79 | −0.46 | 0.054 | −0.41 | 0.09 | −0.18 | 0.28 |
| ADEC-ICF | | | | | | | | | | | | |
| Gr OP-Pr ICF- | −0.33 | 0.055 | −0.38 | 0.054 | −0.15 | 0.69 | −0.23 | 0.35 | −0.4 | 0.1 | −0.34 | 0.044 |
| NRF | | | | | | | | | | | | |
| Gr OP-Pr | −0.38 | 0.024 | −0.48 | 0.013 | −0.08 | 0.82 | −0.41 | 0.08 | −0.4 | 0.1 | −0.28 | 0.1 |
| ADEC-NRF | | | | | | | | | | | | |
| Gr3 OP-Pr | −0.37 | 0.027 | −0.44 | 0.025 | −0.11 | 0.76 | −0.37 | 0.12 | −0.43 | 0.07 | −0.3 | 0.07 |
| ADEC-ICF- | | | | | | | | | | | | |
| NRF | | | | | | | | | | | | |

Chart 3 shows the correlation between the Person Test and the Spearman Test of the Cardiac Index with different thermal spots and gradients, such as: OP4, the average of the hottest spots on each eye, ICF, temperature of the coldest spot in the interciliar region; ADEC, the highest temperature of the region of the dihedral angle formed between the nose and the infraorbitrary region; NRF, the coldest temperature of the region of the root of the nose; NPC, hottest temperature of the region of the tip of the nose; NPF, the coldest temperature of the region of the tip of the nose; Pr ADEC-ICF, the thermal average between both spots; Pr ADEC-NRF, the thermal average between both spots; Pr3 ADEC-ICF-NRF, thermal average between the three spots; Sum X1, the sum of the temperature of the five thermal spots from the face; ICF, the hottest interciliar spot, average interciliar temperature, NRF and average temperature of the root of the nose; Gr, the thermal gradient between both spots; r2 Awake, correlation value in waking patients; r2-MRA, correlation value in patients with mechanical respiratory assistance; r2 VD or nothing, correlation value in patients with vasodilating drugs or without any kind of drug; r2 VC or both, correlation value in patients with vasoconstrictor drugs or VC plus VD; CVP, central venous pressure.

| Pearson's | | | | | | | | | | | | |
|---|---|---|---|---|---|---|---|---|---|---|---|---|
| Test | Blood | | CVP-Blood | | Wedge | | | | | | | |
| Temperature | Pressure | | Pressure | | Pressure | | Heart Rate | | LVSWI | | RVSWI | |
| points | r2 | p | r2 | P | r2 | p | r2 | p | r2 | p | r2 | p |
| OP | 0.22 | 0.19 | 0.02 | 0.86 | 0.18 | 0.31 | -0.06 | 0.72 | 0.04 | 0.82 | 0.11 | 0.55 |
| OP4 | 0.22 | 0.19 | 0.02 | 0.88 | 0.17 | 0.32 | −0.07 | 0.66 | 0.06 | 0.74 | 0.12 | 0.53 |
| ICF | 0.2 | 0.24 | −0.03 | 0.85 | 0.15 | 0.4 | −0.08 | 0.62 | 0.11 | 0.57 | 0.13 | 0.48 |
| ADEC | 0.1 | 0.54 | −0.08 | 0.63 | 0.12 | 0.49 | −0.12 | 0.49 | 0.17 | 0.37 | 0.19 | 0.3 |
| NRF | -0.06 | 0.75 | −0.22 | 0.18 | 0.09 | 0.58 | −0.05 | 0.74 | 0.02 | 0.89 | 0.04 | 0.8 |
| NPC | -0.05 | 0.75 | −0.14 | 0.41 | | | | | | | | |
| NPF | -0.08 | 0.64 | −0.17 | 0.31 | | | | | | | | |
| Pr ADEC-ICF | 0.15 | 0.36 | −0.05 | 0.63 | 0.14 | 0.43 | −0.1 | 0.54 | 0.14 | 0.46 | 0.17 | 0.37 |
| Pr ICF-NRF | 0.04 | 0.82 | −0.16 | 0.34 | 0.13 | 0.48 | −0.07 | 0.68 | 0.06 | 0.75 | 0.08 | 0.65 |
| Pr ADEC-NRF | 0.0006 | 0.99 | −0.18 | 0.29 | 0.11 | 0.5 | −0.08 | 0.63 | 0.08 | 0.66 | 0.11 | 0.57 |
| Pr3 ADEC- | 0.06 | 0.73 | −0.14 | 0.4 | 0.13 | 0.47 | −0.08 | 0.61 | 0.09 | 0.62 | 0.15 | 0.57 |
| ICF-NRF | | | | | | | | | | | | |
| Sumatoria X1 | 0.13 | 0.43 | −0.08 | 0.61 | 0.15 | 0.4 | −0.04 | 0.79 | 0.04 | 0.83 | 0.07 | 0.68 |
| Gr OP-ADEC | 0.22 | 0.19 | 0.23 | 0.17 | 0.1 | 0.57 | 0.13 | 0.45 | −0.28 | 0.13 | −0.19 | 0.31 |
| Gr OP-ICF | 0.019 | 0.91 | 0.13 | 0.42 | 0.03 | 0.8 | 0.06 | 0.7 | −0.14 | 0.44 | −0.05 | 0.77 |
| Gr OP-NRF | 0.24 | 0.15 | 0.32 | 0.06 | 0.003 | 0.98 | 0.02 | 0.86 | 0.0012 | 0.99 | 0.02 | 0.88 |
| Gr OP-Pr | 0.25 | 0.38 | 0.22 | 0.19 | 0.08 | 0.63 | 0.12 | 0.43 | −0.25 | 0.17 | 0.14 | 0.44 |
| ADEC-ICF | | | | | | | | | | | | |
| Gr OP-Pr ICF- | 0.21 | 0.23 | 0.3 | 0.07 | 0.01 | 0.94 | 0.04 | 0.8 | −0.04 | 0.81 | 0.007 | 0.97 |
| NRF | | | | | | | | | | | | |
| Gr OP-Pr | 0.25 | 0.13 | 0.32 | 0.06 | 0.03 | 0.86 | 0.06 | 0.72 | −0.08 | 0.67 | −0.03 | 0.87 |
| ADEC-NRF | | | | | | | | | | | | |
| Gr3 OP-Pr | 0.23 | 0.18 | 0.31 | 0.07 | 0.03 | 0.83 | 0.06 | 0.69 | −0.11 | 0.57 | −0.04 | 0.83 |
| ADEC-ICF- | | | | | | | | | | | | |
| NRF | | | | | | | | | | | | |

Chart 4 shows the correlation between the Pearson Test for different hemodynamic variables, namely: TAM, systemic arterial pressure; TAM-CVP, pressure gradient between both of them; Pw, pulmonary artery occlusion pressure; CF, cardiac frequency; LVSWI, left ventricle systolic work index; SVRWI, right ventricle systolic work index.

| Pearson's test | Systemic Vascular Resistance | | | | | |
|---|---|---|---|---|---|---|
| Temperature | r2-Total | | | | r2- | |
| points | population | p | r2-Awake | p | Sleeping | p |
| OP | −0.29 | 0.08 | −0.025 | 0.9 | −0.91 | 0.0006 |
| OP4 | −0.31 | 0.06 | −0.05 | 0.8 | −0.91 | 0.0006 |
| ICF | −0.35 | 0.036 | −0.07 | 0.73 | −0.93 | 0.0002 |
| ADEC | −0.44 | 0.008 | −0.28 | 0.16 | −0.89 | 0.0012 |
| NRF | −0.35 | 0.037 | −0.28 | 0.16 | −0.91 | 0.0008 |
| NPC | −0.45 | 0.0064 | −0.42 | 0.031 | −0.73 | 0.024 |
| NPF | −0.44 | 0.007 | −0.44 | 0.025 | −0.69 | 0.036 |
| Pr ADEC-ICF | −0.41 | 0.014 | −0.18 | 0.37 | −0.93 | 0.0002 |
| Pr ICF-NRF | −0.37 | 0.025 | −0.22 | 0.28 | −0.93 | 0.0002 |
| Pr ADEC-NRF | −0.4 | 0.016 | −0.29 | 0.14 | −0.9 | 0.0008 |
| Pr3 ADEC-ICF-NRF | −0.4 | 0.016 | −0.24 | 0.23 | −0.93 | 0.0003 |
| Suma X1 | −0.37 | 0.03 | −0.14 | 0.47 | −0.91 | 0.0007 |
| Gr OP-ADEC | 0.32 | 0.053 | 0.45 | 0.021 | −0.19 | 0.61 |
| Gr OP-ICF | 0.17 | 0.32 | 0.11 | 0.57 | 0.12 | 0.75 |
| Gr OP-NRF | 0.24 | 0.16 | 0.31 | 0.12 | 0.62 | 0.07 |
| Gr OP-Pr ADEC- | 0.3 | 0.07 | 0.35 | 0.07 | 0.023 | 0.95 |
| ICF | | | | | | |
| Gr OP-Pr ICF-NRF | 0.25 | 0.14 | 0.28 | 0.16 | 0.36 | 0.33 |
| Gr OP-Pr ADEC- | 0.28 | 0.09 | 0.37 | 0.06 | 0.32 | 0.39 |
| NRF | | | | | | |
| Gr3 OP-Pr ADEC- | 0.28 | 0.09 | 0.34 | 0.08 | 0.24 | 0.52 |
| ICF-NRF | | | | | | |
| Spearman's Test | Systemic Vascular Resistance | | | | | |
| Temperature | r2-Total | | | | r2- | |
| points | population | p | r2-Awake | p | Sleeping | p |
| OP | −0.22 | 0.19 | −0.13 | 0.51 | −0.82 | 0.0058 |
| OP4 | −0.25 | 0.14 | −0.16 | 0.42 | −0.85 | 0.0037 |
| ICF | −0.22 | 0.18 | −0.017 | 0.93 | −0.9 | 0.0009 |
| ADEC | −0.32 | 0.053 | −0.26 | 0.19 | −0.82 | 0.007 |
| NRF | −0.33 | 0.051 | −0.28 | 0.16 | −0.82 | 0.007 |
| NPC | −0.44 | 0.0087 | 0.45 | 0.019 | −0.58 | 0.09 |
| NPF | −0.4 | 0.016 | −0.44 | 0.023 | −0.43 | 0.24 |
| Pr ADEC-ICF | −0.27 | 0.11 | −0.12 | 0.56 | −0.92 | 0.0005 |
| Pr ICF-NRF | −0.28 | 0.09 | −0.19 | 0.33 | −0.92 | 0.0005 |
| Pr ADEC-NRF | −0.32 | 0.056 | −0.25 | 0.21 | −0.82 | 0.007 |
| Pr3 ADEC-ICF-NRF | −0.32 | 0.06 | −0.21 | 0.29 | −0.92 | 0.0005 |
| Suma X1 | −0.26 | 0.12 | −0.11 | 0.58 | −0.92 | 0.0005 |
| Gr OP-ADEC | 0.3 | 0.07 | 0.4 | 0.039 | −0.28 | 0.46 |
| Gr OP-ICF | 0.08 | 0.6 | −0.004 | 0.98 | 0.16 | 0.67 |
| Gr OP-NRF | 0.28 | 0.09 | 0.31 | 0.11 | 0.4 | 0.28 |
| Gr OP-Pr ADEC- | 0.26 | 0.12 | 0.27 | 0.17 | 0.016 | 0.96 |
| ICF | | | | | | |
| Gr OP-Pr ICF-NRF | 0.25 | 0.13 | 0.26 | 0.19 | 0.18 | 0.63 |
| Gr OP-Pr ADEC- | 0.32 | 0.06 | 0.38 | 0.052 | 0.06 | 0.86 |
| NRF | | | | | | |
| Gr3 OP-Pr ADEC- | 0.29 | 0.08 | 0.32 | 0.11 | 0.26 | 0.6 |
| ICF-NRF | | | | | | |

Chart 5 shows the correlation between Pearson Test and Spearman Test for the Systemic Vascular Resistance (SVR).

Thus, it was found that only a few thermal spots of the face can suitably predict the hemodynamic variables of interest, i.e. CI and SVR, the same occurs with the thermal gradients. Likewise, it was found that the thermal spots and gradients to be taken into account will be different according to the specific situation of the patient: whether the patient is asleep or awake, under the influence of vasodilating or vasoconstrictor drugs, the PVC values and gender of the patient.

In conclusion, it could be said that specific thermal spots and gradients exist in the face of the patients and these spots and gradients are directly and indirectly correlated with CI and SVR.

Both the aforementioned FIRST and SECOND investigations prove the existence of specific spots on the skin of the face and the hand, as well as thermal gradients, which are related to cardiovascular performance. The best and highest correlations as valuated by the Spearman and Pearson Tests were employed for the computational algorithms, based on ML techniques for the development of the two modalities of the invention.

### Function, Modality A

The invention described herein, by capturing thermal images from the face and the hand of the patient with any camera that can measure infrared radiation, allows for the prediction of the distance (in meters) which would be completed by the patient if subjected to a 6MWT. This method (6MWT) could be considered as a surrogate for detailed assessment of the reaction of the respiratory, cardiovascular, metabolic, musculoskeletal and neurosensorial systems of the patient subjected to the strain caused by physical exercise. As a consequence, it is one of the most utilized tools for this purpose in individuals with cardiac and respiratory diseases.

With the object of replacing tests such as the aforementioned, and with the purpose of reducing the time taken for testing the patient without the necessity for specialized personnel and with the capacity to perform it at any time or place, the invention evaluates a series of thermal parameters which, along with other general data from the patient, brings high-correlativity results in a simple and immediate fashion.

The computational algorithm analyses an average of three thermal photos from the front and profile of the face and the palm of the hand of the patient to be tested.

Hot and cold spots identified in the thermal images are employed. These thermal spots were located and studied following scientific investigation of the aforementioned first and second modalities. The thermal data of spots and gradients is integrated for the analysis along with data of clinical and non-clinical values also previously tested and selected, such as age, gender, size, weight, cardiac frequency, oxygen saturation, PH type, humidity and room temperature.

The thermal spots employed are the following:
Head, front: the hottest spot in both ocular regions and the coldest spot of the nasal region.
Head, profile: the hottest spot obtained from the area of the external auditive conduct, hottest spot in the Pterion region and the coldest obtained spot from the auricular pavilion.

Palm of the hand: hottest spot of the wrist, hottest spot of the center of the palm, hottest spots from the five fingertips.

The program employs a predictive model, which was developed employing ML techniques that allow us to establish the distance that could be walked by the patient if subjected to a 6MWT, expressed in meters.

In this case, after analyzing and employing several algorithms, the final algorithm selected to analyze the data and predict new entries was ‘Fast Tree Regression Binary’. With this algorithm it was possible to obtain the best result from the set of analyzed data.

The tool and library employed to perform these calculations was Microsoft ML (ML.NET Model Builder—https://dotnet.microsoft.com/apps/machinelearning-ai/ml-dotnet/model-builder). This tool and library allow an agile analysis with ‘Visual Studio’, since it contains tools which allow us to obtain faster results with the available algorithms. The best result was obtained with the following parameters:
Number of leaves: 7
Minimum Example Count Per Leaf: 10
Number of Trees =100
Learning Rate: 0.1602678f
Shrinkage: 0.6471589f
Bagging Example Fraction: 0
Best Step Ranking Regression Trees: true

These parameters are normally provided by the library, but in some cases, we've modified them in order to obtain a better prediction. These are the libraries used to develop these modules:
Microsoft.ML, version: 1.3.1
Microsoft.ML.FastTree, version: 1.3.1

This is the main piece of code to resolve this module (to see with a better format, see the FIG. 16):

```
public static IEstimator<ITransformer>
BuildTrainingPipeline (MLContext mlContext)
{
  / / Data process configuration with pipeline data
transformations
  var inputColumnNames = ColumnNames ForModel Input ( );
  var dpp =
mlContext.Transforms.Conversion.ConvertType (new[ ] { new
InputOutputColumnPair (“HTA”, “HTA”), new
InputOutputColumnPair (“HTA_DBT”, “HTA_DBT”), new
InputOutputColumnPair (“DBT”, “DBT”), new
InputOutputColumnPair (“IFDE_5”, “IFDE_5”), new
InputOutputColumnPair (“PG”, “PG”), new
InputOutputColumnPair (“BLOQ_CA”, “BLOQ_CA”), new
InputOutputColumnPair (“IECA_ARA2”, “IECA_ARA2”) })
.Append (mlContext.Transforms.Categorical.OneHotEncoding (new [ ]
{ new InputOutputColumnPair (“Sexo”, “Sexo”), new
InputOutputColumnPair (“SujetoDeEstodio”, “SujetoDeEstodio”),
new InputOutputColumnPair (“TipoHP”, “TipoHP”), new
InputOutputColumnPair (“ERA”, “ERA”) }))
.Append (mlContext.Transforms.Concatenate (“Features”,
inputColumnNames));
  / / Set the training algorithm
  var trainer = mlContext.Regression.Trainers.FastTree (new
FastTreeRegressionTrainer.Options ( ) {
    NumberOfLeaves = 7,
    MinimumExampleCountPerLeaf = 10,
    NumberOfTrees = 100,
    LearningRate = 0.1602678f,
    Shrinkage = 0.6471589f,
    LabelColumnName = “CF”,
    FeatureColumnName = “Features”,
    BaggingExampleFraction = 0,
    BestStepRankingRegressionTrees = true, / /un poco mejor
  });
  var trainingPipeline = dpp.Append (trainer);
  return trainingPipeline;
}
```

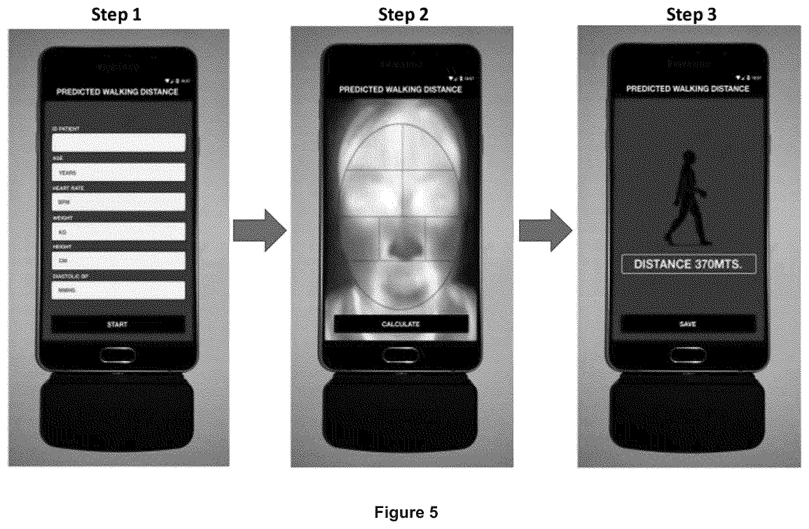

The obtained model was employed to evaluate data from new testing. It can be employed for applications in different platforms, such as mobile devices, the web, etc. By uploading the aforementioned data, an immediate result is obtained (see FIG. 5, where the series of steps and the normal use of the device, which consists of a Samsung A5 cellphone with a FLIR ONE thermal camera attached, is shown schematically).

### Function, Modality B

By employing the same principle as explained previously, the invention can estimate the CI and the SVR of a patient, when knowledge of the hemodynamic state is required, without resorting to an invasive method such as, for example, the Swan-Ganz catheter.

The thermal spots of reference were found through testing hospitalized patients in a Critical Care Unit. They required invasive hemodynamic monitoring employing the Swan-Ganz catheter, and more than 17 thermal spots of their faces were tested, along with averages of 7 anatomic regions, 3 different ways of averaging the distinctive thermal spots and 15 distinctive thermal gradients.

The thermal values that showed a better result when correlated with the CI and the SVR obtained through Swan-Ganz were integrated for analysis along with data from clinical and non-clinical values, already tested in anticipation and selected throughout the trials and research previously mentioned. This additional data encompasses: gender; exact PVC value as measured by a central venous catheter or approximation, demonstrated through the display of the external jugular vein ingurgement; usage or not of vasodilating or vasoconstrictor intravenous drugs; waking state of the patient—whether the patient is awake or pharmacologically asleep; humidity and room temperature.

The thermal spots employed in the computational algorithm for prediction are: the spot with the highest temperature in the interciliar region (ICF), the spot with the lowest temperature in the region of the root of the nose (NRF), the spot with the highest temperature of the inferior part of the dihedral angle, formed by the nasal wing and the infraorbitrary region (ADEC), and the averages and gradients between them.

The ‘Modality B’ of the invention avoids the employment of invasive methods such as a catheter in a pulmonary artery, intra-arterial catheters, etc., that imply risks for the patient and are complex in their implementation, since they demand expertise and experience from the professionals. The ‘Modality B’ is a fast and simple method that doesn't demand qualified personnel; moreover, it is harmless since it is a non-invasive method. It can be performed at any moment, anywhere.

The ‘Modality B’ employs thermal and non-thermal data (as previously explained), integrating them in two predictive models which are different from those of Modality A, which were developed employing ML techniques.

Utilizing the same tools for analysis as Modality A, a new data set was considered. In this case a classifying algorithm was employed for the analysis of prediction of the new data set.

In this set of data, the objective was to predict the Cardiac Index and Systemic Vascular Resistance. It is worth mentioning that both variables to be predicted were calculated as binary variables; the cut-off points taken into account were: CI 2.5 liters/min/m² and SVR 1000 dines/sec.cm⁻⁵.

For the first step, Cardiac Index, the algorithm that provided the best results was ‘One-Versus-All (OVA)’. This algorithm, with the libraries employed, required a standardized parametrization, describing the data entry and the data to be predicted.

For the second case, Systemic Vascular Resistance, the algorithm that got closer to predicting the results was ‘Light Gbm’.

These are the libraries used to develop these modules:
Microsoft.ML, version: 1.5.0-preview
Microsoft.ML.FastTree, version: 1.5.0-preview
Microsoft.ML.LightGBM, version: 1.5.0-preview

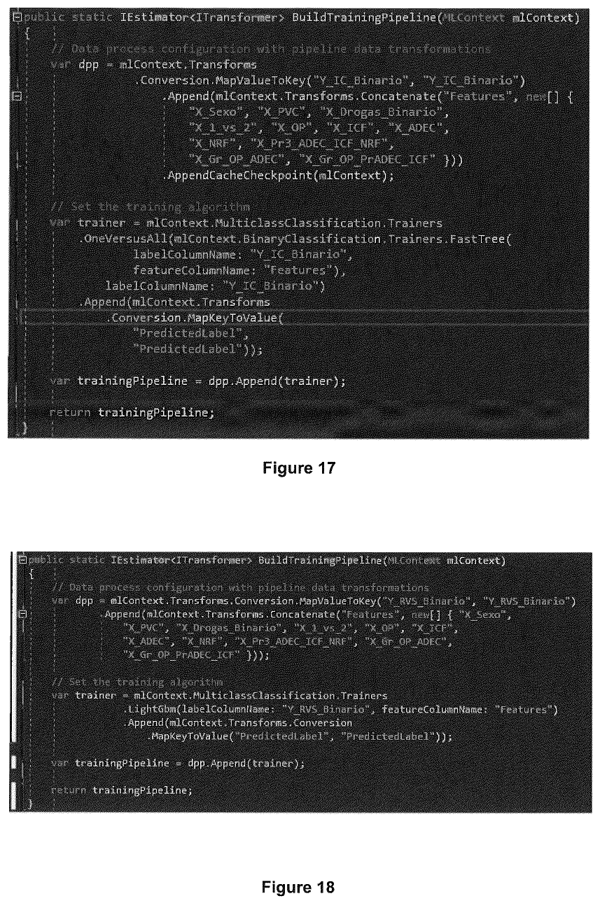

These are the two main pieces of code to resolve these modules:
For One-Versus-All (OVA). This is the main piece of code to resolve this module (to see with a better format, see the FIG. 17):

```
public static IEstimator<ITransformer>
BuildTrainingPipeline (MLContext mlContext)
{
  // Data process configuration with pipeline data
transformations
  var dpp =
mlContext.Transforms.Conversion.MapValueToKey (“Y_IC_Binario”,
“Y_IC_Binario”)
.Append(mlContext.Transforms.Concatenate (“Features”, new [ ] {
“X_Sexo”, “X_PVC”, “X_Drogas_Binario”, “X_1_vs_2”, “X_OP”,
“X_ICF”, “X_ADEC”, “X_NRF”, “X_Pr3_ADEC_ICF_NRF”,
“X_Gr_OP_ADEC”, “X_Gr_OP_PrADEC_ICF” }))
.AppendCacheCheckpoint (mlContext);
  // Set the training algorithm
  var trainer =
mlContext.MulticlassClassification.Trainers.OneVersusAll (mlCon
text.BinaryClassification.Trainers.FastTree (labelColumnName:
“Y_IC_Binario”, featureColumnName: “Features”) ,
labelColumnName: “Y_IC_Binario”)
.Append (mlContext.Transforms.Conversion.MapKeyToValue (“Predict
edLabel”, “PredictedLabel”));
  var trainingPipeline = dpp.Append (trainer);
  return trainingPipeline;
}
```

For Light Gbm. This is the main piece of code to resolve this module (to see with a better format, see the FIG. 18):

```
public static IEstimator<ITransformer>
BuildTrainingPipeline (MLContext mlContext)
{
  // Data process configuration with pipeline data
transformations
  var dpp =
mlContext.Transforms.Conversion.MapValueToKey (“Y_RVS_Binario”,
“Y_RVS_Binario”)
.Append(mlContext.Transforms.Concatenate (“Features”, new [ ] {
“X_Sexo”,
      “X_PVC”, “X_Drogas_Binario”, “X_1_vs_2”,
“X_OP”, “X_ICF”,
      “X_ADEC”, “X_NRF”, “X_Pr3_ADEC_ICF_NRF”,
“X_Gr_OP_ADEC”,
      “X_Gr_OP_PrADEC_ICF” }));
  / / Set the training algorithm
  var trainer = mlContext.MulticlassClassification.Trainers
      .LightGbm (labelColumnName: “Y_RVS_Binario”,
featureColumnName: “Features”)
      .Append (mlContext.Transforms.Conversion
        .MapKeyToValue (“PredictedLabel”,
“PredictedLabel”));
  var trainingPipeline = dpp.Append (trainer);
  return trainingPipeline;
}
```

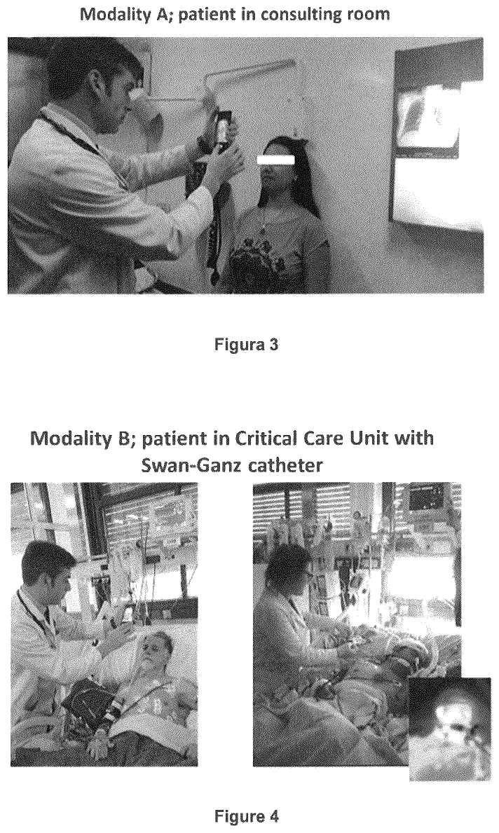

By doing so, it allows us to establish (1) CI and (2) SVR from the patient to be tested in a simple, safe and immediate way. FIG. 4 exemplifies the method of use.

Obtained results—The demonstrated value of the technology in different health care environments and different interested parties, as appropriate.

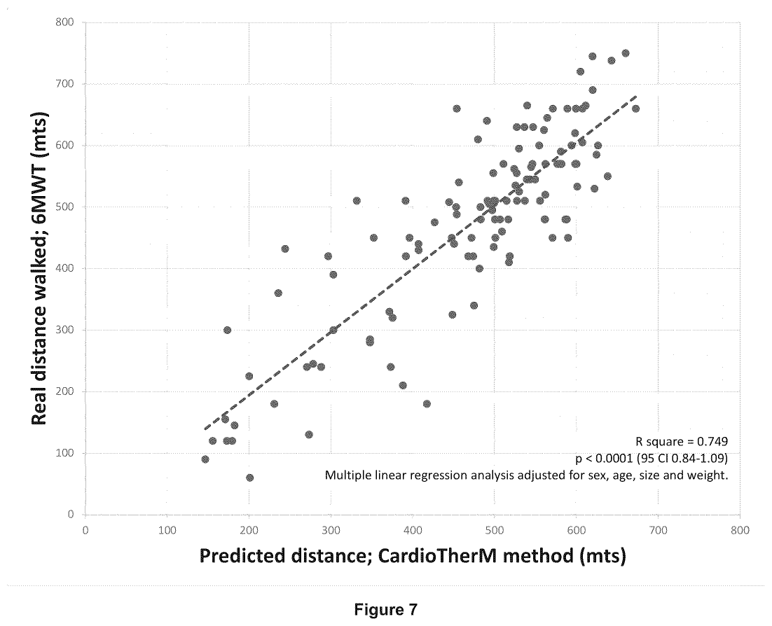

“Modality A”: a prospective validation was performed, 6MWT versus invention, “Modality A”, in controls and patients with Pulmonary Hypertension (PH); 121 cases. The multiple lineal regression analysis showed a statistically significant R square (See FIG. 7).

The Bland-Altman plot showed a good agreement between the 6MWT and invention, “Modality A”. Sensitivity 91.3%, specificity 89.8%, PPV 67.7% and NPV 97.7%.

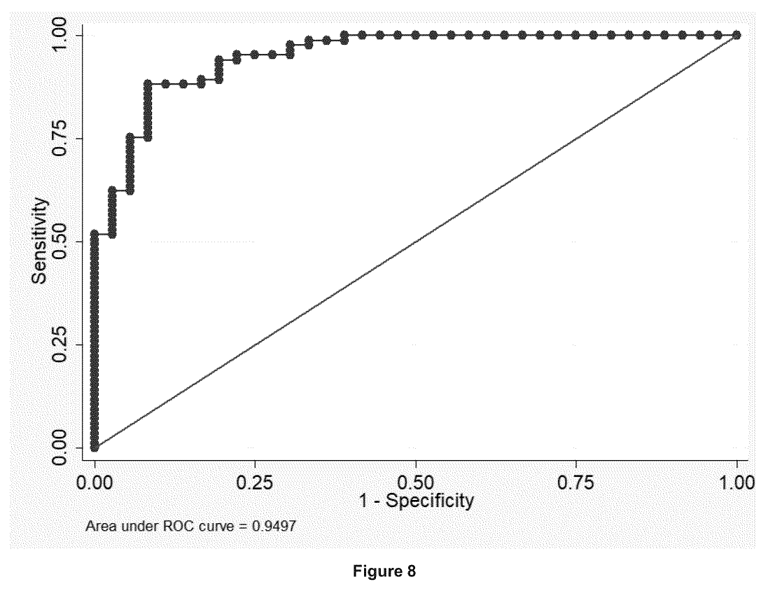

In patients who walk less than 440 meters, the area under the ROC curve shows a compelling diagnostic performance of the test and high accuracy (ROC curve=0.9497). (Refer to FIG. 8).

This is important, because the value of 440 meters is a breakpoint between a good and a bad prognosis for patients with PH.

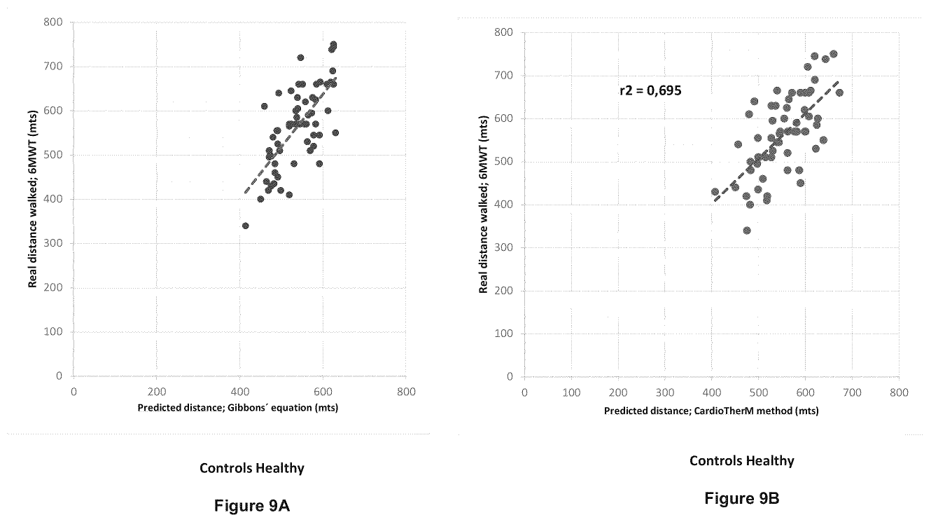

When the predictive power of the invention, “Modality A” is compared versus the Gibbons equation, which is based on age, weigh, high, gender, which is usually used to predict walking distance in healthy people, it was found that in healthy patients, the correlation was very similar to that shown in the Gibbons plot. (Refer to FIG. 9).

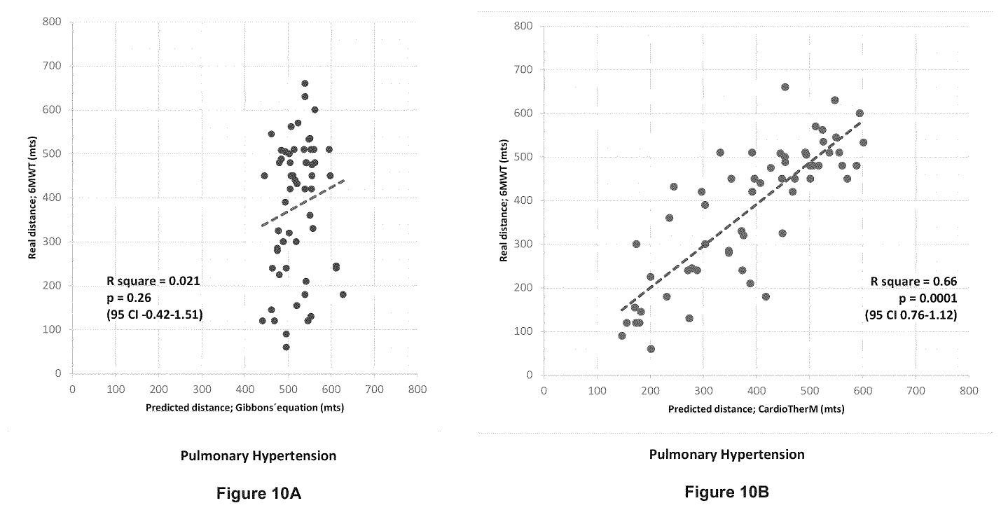

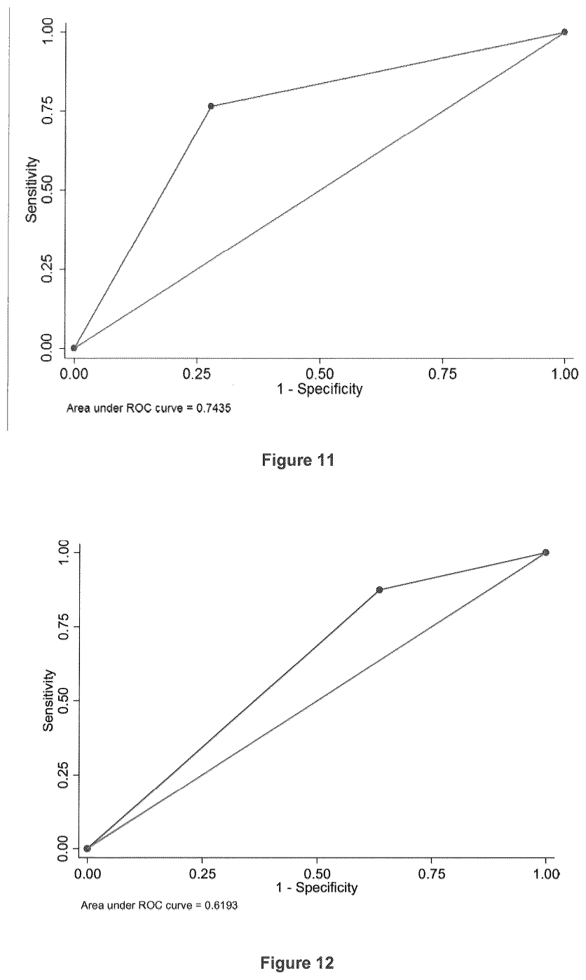

However, when patients with PH are analyzed and the predictive equation of Gibbons is applied, then the correlation is less accurate. In contrast, when the invention is used, “Modality A”, the correlation is much higher, being that of 0.66. (See FIG. 10).

“Modality B”: The properties of the invention, “Modality B”, were calculated in values of sensitivity, specificity, negative predictive value and positive predictive value to detect heart failure, using the Swan-Ganz catheter and thermodilution as reference methods.

Sensitivity, Specificity, Positive Predictive Value, and Negative Predictive Value were calculated. The Receiver Operating Characteristic (ROC) curve of the aforementioned method was also plotted. Heart failure was defined as a CI<2.5 liter/min/m² measured by thermodilution method and Swan-Ganz catheter. A value of p<0.05 was considered significant. A transverse-sectional and prospective recruitment study was conducted. 35 hemodynamic measurements were conducted in 18 patients who were under hospitalization in a Cardiovascular Critic Care Unit for different reasons which required invasive hemodynamic monitoring, using Swan-Ganz catheter.

The usual invasive hemodynamic variables were recorded including CI, SVR and PVC. The “Modality B” test was performed at the same time as each thermodilution measurement using a Swan-Ganz catheter.

The results were as follows: at the moment of taking measurements by Swan-Ganz, 52% of the patients suffered from cardiac insufficiency and 49% at the moment of employing the invention, ‘Modality B’. The invention, ‘Modality B’ presented a Reliability (Accuracy)=72.2% for testing cardiac insufficiency.
Sensitivity=72.2 (CI95% 51.5 to 92.8)
Specificity=72.2 (CI95% 51.5 to 93.2)
VPP=72.2 (CI95% 51.5 to 92.8)
VPN=57 (CI95% 20.3 to 93.7)
Area under the ROC curve 0.619 (See FIG. 12)

### Practical Implementation

It is a surrogate method to measure the hemodynamic state and the effort capacity of the patient, the result is immediate, easy to use at any time and any place, and it does not require skill in its use, which allows it to be used by any professional, nurse or health technician.

Its versatility would allow it to be used, under appropriate conditions, both in Critical Care Units, outpatient medical offices and even by the patient themselves at home subject to the availability of the right elements and under the right conditions.

It could avoid the need for the use of invasive methods and their complications by being used as a screening method in patients where there are doubts about whether or not to use invasive methods.

### Additional Utilities

1. The invention can be correlated to invasive methods, it can predict CI and SVR. But by modifying the employed computational algorithm it could predict the congestive state of a patient with cardiac failure in the right or left ventricle, since we find statistically significant correlations with the Pearson and Spearman Tests between certain thermal spots and gradients, with the PVC value measured through an invasive method such as Swan-Ganz catheter.
2. Considering the characteristics of the method and the physiological principle of its function, the invention could have its own predictive value for mortality or morbimortality, independently from that of the traditional testing methods, for example, the Cardiopulmonary Effort Test with measurement of oxygen consumption. For this purpose, it requires testing in a prospective trial with the appropriate sample quantity.
3. The computational algorithm for evaluation and analysis of thermal images can be used in telemedicine. This allows patients treated or studied in one place to be treated or tested elsewhere by another health care professional who is not physically present with the patient. This also allows patients to be evaluated at home and without medical presence, a method called “selfie mode”.
4. By means of testing of a larger number of individuals (patients), the invention could have a more suitable algorithm, which would allow it to predict an approximate value of CI and SVR as a continuous variable, and not only provide a dichotomous result of ‘sick’ or ‘healthy’, ‘high’ or ‘low’ value.

### IMPLEMENTATION EXAMPLES

### Clinical Case 1; Patient L. G.:

Male, 71, undergoing hospitalization in Coronary Unit due to Cardiogenic Shock in context of valvular dilated myocardiopathy with severe left ventricle function deterioration. This required invasive hemodynamic management, with Swan-Ganz catheter and treatment with inotropes and vasodilators.

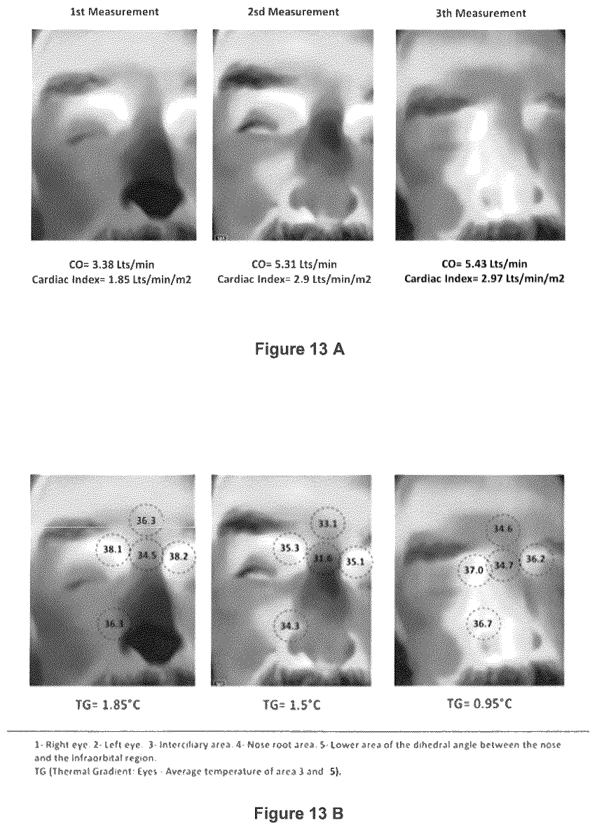

Measurements were made with the invention according to Modality B, prior to measurement by thermodilution with a Swan-Ganz catheter, at three different points in its evolution. (see FIG. 13A and FIG. 13 B).

### 1^{st} Measurement:

Data involved: room temperature 22° C., room humidity 57%; PVC Value 11 mmHg; Male; waking state; Vasoactive drugs employed: dobutamine and milrinone.
Result according to the method of the invention, Modality B, patient with heart failure.
Result according to the Swan-Ganz method: CI=1.85 liters/min/m².

### 2^{nd} Measurement:

Data involved: room temperature 22.1° C.; ambient humidity 55%; PVC value 7 mmHg; Male; Waking state; Vasoactive drugs used: dobutamine, milrinone and sodium nitroprusside.
Result according to the method of the invention, Modality B: patient without heart failure.
Result according to Swan-Ganz: IC=2.9 liters/min/m².

### 3^{rd} Measurement:

Data involved: room temperature 22.5° C.; ambient humidity: 58%; PVC Value 6mmHg; Male; Waking state; Vasoactive drugs used: dobutamine, milrinone and sodium nitroprusside.
Result according to the method of the invention, Modality B: patient without heart failure.
Result according to Swan-Ganz: CI=2.97 liters/min/m².

### Clinical Case 2; Patient H. B.:

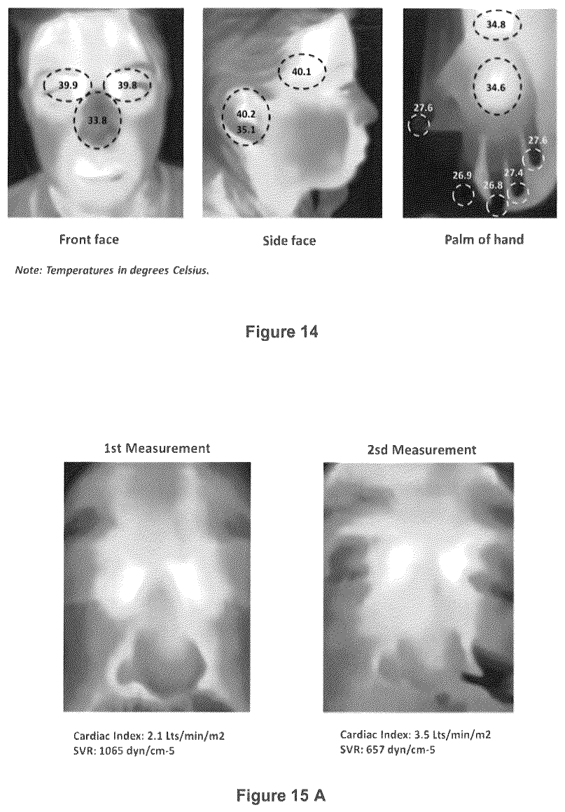

25-year-old female, carrier of Group 1 Pulmonary Hypertension, idiopathic, in functional class 2, NT-proBNP 839 pg/ml, with a weight of 63 kg and height of 165 cm. Treated with a specific combined therapy for Pulmonary Hypertension: sildenafil, macitentan and treprostinil. (See FIG. 14).

Data involved according to the method of the invention, Modality A: Weight 63 kg.; Height—165 cm; Sex—Female ; Age—25 years; PH Type: idiopathic; Functional Class 2; Treatment with 3 particular drugs; Oxygen saturation in blood 97% and Cardiac frequency 78×min; Systolic Arterial pressure 112 mmHg, diastolic: 76 mmHg; Room temperature 24.7° C.; ambient humidity 61%.

### Results

Predicted distance by Gibbons equation=554 m
Real distance walked in 6MWT=450 m
Predicted distance according to the method of the invention, Modality A=462 m

### Clinical Case 3; Patient C. C. S.:

62-year-old man, undergoing hospitalization in Coronary Unit due to Cardiogenic Shock in the context of post-operative cardiac surgery with severe left ventricular function deterioration. Invasive hemodynamic monitoring with Swan-Ganz catheter and treatment with inotropics and vasoconstrictors were required.

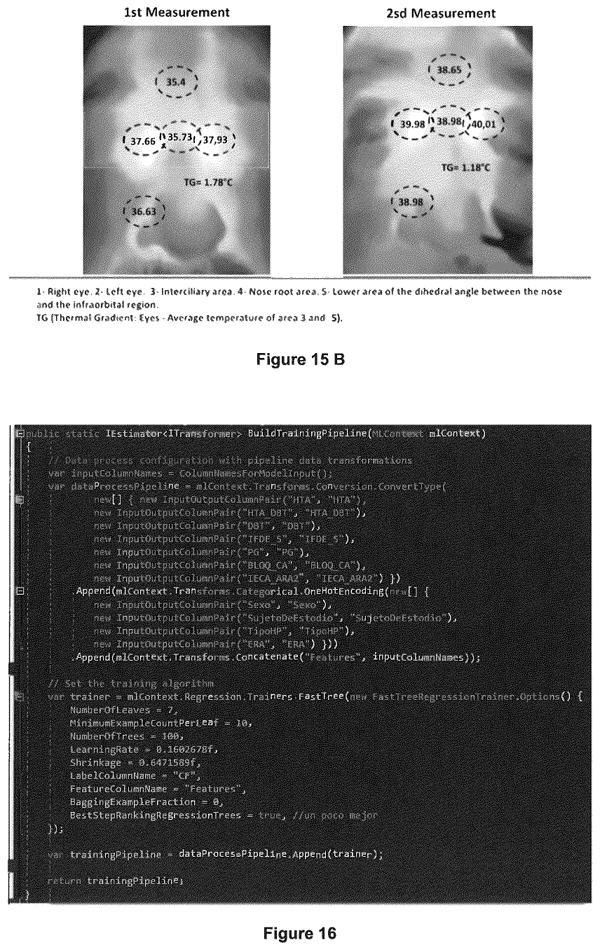

Measurements were taken with the invention according to Modality B, before proceeding with measurements by thermodilution with a Swan-Ganz catheter, in two different moments of its process. (See FIG. 15A and FIG. 15B).

### 1^{st} Measurement:

Data involved: Room temperature 23.7; Ambient humidity 38%; PVC value 9 mmHg; male; state of pharmacological sedation in ARM; vasoactive drugs used: dobutamine, norepinephrine, epinephrine, phenylephrine, vasopressin and methyl blue.
Result according to the method of the invention, Modality B, patient with heart failure. High SVR.
Result according to Swan-Ganz: CI=2.1 liters/min/m². SVR=1065 dines/sec.cm⁻⁵.

### 2^{nd} Measurement:

Data involved: room temperature 26.1; ambient humidity 26%; PVC value 11 mmHg; Male; State of pharmacological sedation in ARM; Vasoactive drugs used: norepinephrine and epinephrine.
Result according to the method of the invention, Modality B, patient without heart failure. Reduced SVR.
Result according to Swan-Ganz: CI=3.5 liters/min/m², SVR=657 dines/sec.cm⁻⁵.

1. (canceled)

2. (canceled)

3. (canceled)

4. (canceled)

5. (canceled)

6. (canceled)

7. (canceled)

8. (canceled)

9. (canceled)

10. A non-invasive diagnostic method of diagnosing cardiovascular performance by clinical testing to determine the hemodynatnic state and the effort capacity of an individual subjected to trial, the method comprising the steps of:
capturing thermal images utilizing an infrared thermographic camera from one of a) a face and a hand of the individual, and b) a front and lateral side of the face of the individual;
entering the thermal images on a magnetic medium to be analyzed;
on the captured thermal images, determining temperature values of a series of specific preselected points in established regions of the individual;
analyzing said temperature values through a library of code and software of the infrared thermographic camera, which allows an interpretation of infrared color spectrum and translation into temperature in degrees centigrade;
entering on a magnetic medium additional data consisting of general parameters of the individual and the environment in which the method is taking place; and
analyzing the acquired data obtained through the previous steps by means of predictive Machine Learning (ML) techniques in order to predict the results of:
i) a distance walked by the individual if subjected to a Six-Minute Walk Test (6MWT), or
ii) a Cardiac index (CI) and Systemic Vascular Resistance (SVR) of the individual if subjected to hemodynamic monitoring with Swan-Ganz catheter, if the individual is subjected to either of tests i) and ii).

11. The method of claim 10, wherein the temperature values are determined through thermal images from the front and the lateral side of the individual's face, and the palm of the individual's hand.

12. The method of claim 11, wherein the temperature values are determined from the captured thermal images in regions of the front and lateral side of the individual's face selected from a left eye, a right eye, a nose, an interciliar region, a dihedral angle formed by the nose and the infraorbital region, an ear canal and a pinna, and in regions of the individual's palm of a hand selected from a wrist, a central region of the palm and fingertips.

13. The method of the claim 12, wherein only a hottest point and a coldest point of each region are considered to determine the temperature values.

14. The method of the claim 13, wherein the hottest points are elected from the left eye (Oi), the right eye (Od), a tip of the nose (Na), the dihedral angle formed between the nose and infraorbital region (ADEC), the ear canal (Ca), and the pinna (Pa), the wrist, the central region of the palm of the hand and the fingertips.

15. The method of the claim 13, wherein the coldest points from the nose (NRF) and the interciliar regions (ICF).

16. The method of claim 1.3, wherein when the 61MWT is predicted, thermal gradients are established a) between the temperature values of the right eye and the nose (NO gradient), b) between the temperature values of ear canal and the pinna (CP gradient), and c) between the temperature values of distinctive points of the hand.

17. The method of claim 13, wherein when the CI and SVR are predicted, thermal averages are established a) between the ICF, the NRF and the ADEC, b)at the OP, and c) between ADEC and ICF.

18. The method of the claim 10, wherein the general parameters from the individual are at least one of a gender, a size, a weight and a cardiac frequency, a blood pressure, a functional class, a vasodilator drugs the patient is taking and an oxygen saturation.

19. The method of the claim 10, wherein the general parameters from the individual are at least one of a gender, a use of intravenous vasoconstrictor or vasodilator drugs, an awake or an asleep state, and an approximative value of central venous pressure (PVC) in the right atrial.

20. The method of the claim 10, wherein the additional general parameters from the individual are at least one of gender, use of intravenous vasoconstrictor or vasodilator drugs, awake or asleep state, and an approximative value of central venous pressure (PVC) in the right atrial.
